(19) United States
(12) Patent Application Publication
(10) Pub. No.: US 2024/0335197 A1
(43) Pub. Date: Oct. 10, 2024

(54) ACTIVE BENDING SYSTEM OF STAPLER

(71) Applicant: Suzhou Meidong Huicheng Precision Components Co., Ltd., Suzhou, Jiangsu (CN)

(72) Inventors: SHITAO MA, Suzhou, Jiangsu (CN); CHAOYANG WEN, Suzhou, Jiangsu (CN); GAN ZHANG, Suzhou, Jiangsu (CN); GAOJU LI, Suzhou, Jiangsu (CN)

(21) Appl. No.: 17/797,298

(22) PCT Filed: Nov. 9, 2021
(86) PCT No.: PCT/CN2021/129623

§ 371 Date: Mar. 20, 2024

(30) Foreign Application Priority Data
Aug. 9, 2021 (CN) 202110906110.4

(51) Int. Cl. A61B 17/072

(52) U.S. Cl. CPC A61B 17/07207; A61B 2017/07257; A61B 2017/07285

(57) ABSTRACT

The present disclosure provides an active bending system of a stapler, relating to the technical field of medical equipment. The active bending system of the stapler comprises an anvil, steering pull rods, an inner sleeve, a sleeve connector, an outer sleeve, a connecting piece, a steering control seat, a steering conversion seat, a steering linkage and a cutting mechanism, wherein the inner sleeve is arranged in the outer sleeve for limiting other parts; the steering pull rods are connected to a right side of the inner sleeve for connecting the inner sleeve and components in a stapler body; the steering conversion seat is movably hinged in the steering pull rods for pushing or pulling back the steering pull rods to move left and right.

### TECHNICAL FIELD

The present disclosure belongs to the technical field of medical equipment and specifically relates to an active bending system of a stapler.

### BACKGROUND

The stapler is the first stitching instrument in the world. It has been used for gastrointestinal anastomosis for nearly a century. It was not until 1978 that tube-type staplers were widely used in gastrointestinal surgery. It is generally divided into one-time or reusable staplers, imported or domestic staplers. The stapler is a medical substitute for traditional manual suture. Because of the development of modern science and technology and the improvement of manufacturing technology, the stapler for clinical use is reliable in quality, convenient in use, and appropriate in tightness, especially with advantages of rapid suture, simple operation, few side effects and surgical complications, etc. It also allows surgical excision of previously unresectable tumors.

### SUMMARY OF INVENTION

### Technical Matters

At present, the bending system of the straight-line cutting stapler in the market can be roughly divided into two types: one is the adaptive passive bending system manufactured by Johnson & Johnson, and the other is the single pull rod active bending system invented by Techwise. However, both bending systems have insufficient bending angle accuracy and stability, reducing doctor experience and surgical efficiency and success rate.

### Solution of Technical

### SUMMARY

The object of the present disclosure is to provide an active bending system of a stapler, aiming at solving the problems of insufficient bending angle accuracy and stability, reduced doctor experience and surgical efficiency and success rate in the existing two types of bending systems of the straight-line cutting stapler: one is the adaptive passive bending system manufactured by Johnson & Johnson, and the other is the single pull rod active bending system invented by Techwise.

To achieve the above objective, the present disclosure provides the following technical solutions:

An active bending system of a stapler comprises an anvil, steering pull rods, an inner sleeve, a sleeve connector, an outer sleeve, a connecting piece, a steering control seat, a steering conversion seat, a steering linkage and a cutting mechanism, wherein the inner sleeve is arranged in the outer sleeve for limiting other parts; the steering pull rods are connected to a right side of the inner sleeve for connecting the inner sleeve and components in a stapler body; the steering conversion seat is movably hinged in the steering pull rods for pushing or pulling back the steering pull rods to move left and right; the steering linkage is arranged on the steering conversion seat for connecting a steering power element of the stapler body and the steering conversion seat to provide power for a bending function of the stapler; there are two steering pull rods which are movably hinged on upper and lower sides of the steering conversion seat respectively for pulling the steering control seat to move left and right to realize a bending function of a stapler head; the steering control seat is connected to the other end of the two steering pull rods, and the steering control seat is rotationally connected to the inner sleeve through a limit shaft for circumferential motion around the limit shaft to realize bending; a steering sleeve is arranged on the outer sleeve for limiting the anvil and a loading unit base, to provide the stapler with initial clamping force to clamp tissue; and the anvil and the loading unit base are connected to a left side of the outer sleeve through the steering sleeve, to provide the stapler with functions of tissue clamping and titanium pin forming.

Preferably, the cutting mechanism comprises a cutter assembly, an inner sleeve cover, a trigger shaft, and a propulsion rack; the cutter assembly, the inner sleeve cover, the trigger shaft and the propulsion rack are arranged in sequence from left to right; the inner sleeve cover is clamped on the inner sleeve for limiting the cutter assembly and the trigger shaft to ensure smooth operation; the cutter assembly is arranged on a left side of the inner sleeve cover for realizing a cutting function of the stapler and pushing the titanium pin to penetrate the tissue; the trigger shaft is connected to a right side of the cutter assembly for pushing or pulling back the cutter assembly; and the propulsion rack is connected to a right side of the trigger shaft for providing power for forward and backward movement of the cutter assembly with a power gear connected to the stapler body and the trigger shaft.

Preferably, two steering connecting pins are fixed on the steering control seat, and the two steering pull rods are movably hinged with the steering control seat through the two steering connecting pins respectively.

Preferably, a cutter steering seat is arranged in the inner sleeve, and the cutter steering seat matches with the cutter assembly.

Preferably, the anvil is rotationally connected with the steering control seat through a connecting pin, and the loading unit base is fixed with the steering control seat.

Preferably, a connection between the two steering pull rods and the steering conversion seat is of a waist-shaped hole structure.

### BENEFICIAL EFFECTS OF INVENTION

### Beneficial Effect

Compared with the prior art, the present disclosure has the following beneficial effects:

The bending system can realize fixed-angle bending or arbitrary-angle bending according to different structures or transmission systems of the stapler body, and can adjust angles flexibly. The bending system adopts a symmetrical design with two pull rods, and the connection between the two steering pull rods and the steering conversion seat is of the waist-shaped hole structure, wherein only one steering pull rod is used to pull the steering conversion seat during bending or reset, and there are advantages of simple assembly and connection and symmetrical limit clearance. The steering conversion seat moves flexibly and stably in the waist-shaped hole area, without resistance in cyclical switching of bidirectional driving and interference in linear movement with each other. One steering pull rod generates thrust and the other steering pull rod generates tension, and the cooperation of thrust and tension makes the steering control seat complete bending movement and enables more accurate angle adjustment. The system is designed to pull the steering linkage to control the steering control seat for active bending, effectively improving operation convenience, flexibility and stability.

### DESCRIPTION OF THE DRAWINGS

### Brief Description of Drawings

Drawings are an integral part of the Specification and for further understanding of the present disclosure. Drawings are for, together with the embodiments of the present disclosure, interpreting the present disclosure, and are not restrictions to the present disclosure. In which:

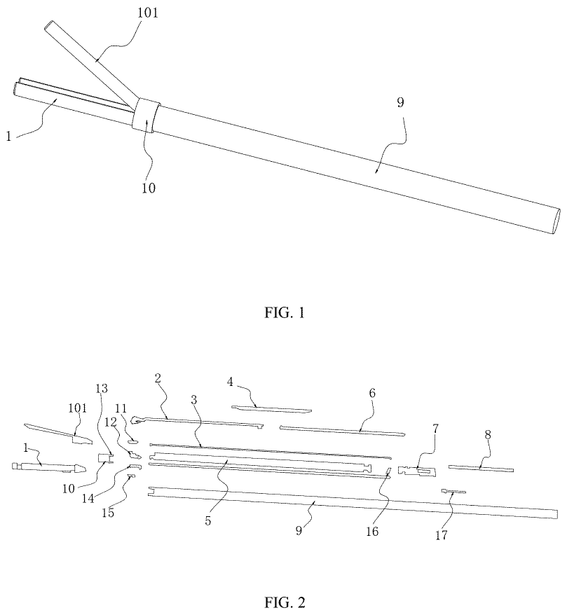

FIG. 1 is a structure diagram of the present disclosure;

FIG. 2 is an exploded view of the present disclosure;

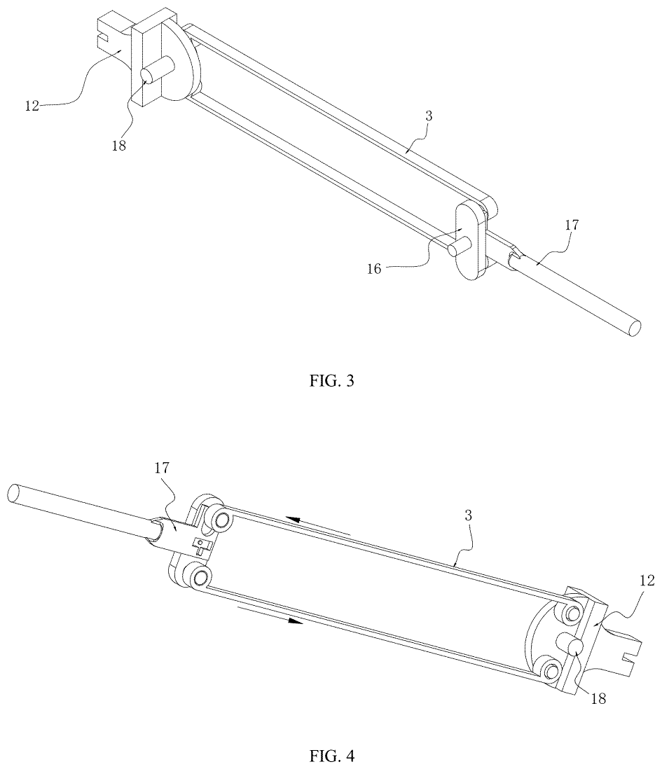

FIG. 3 is a first view of a partial view of the present disclosure;

FIG. 4 is a second view of the partial view of the present disclosure;

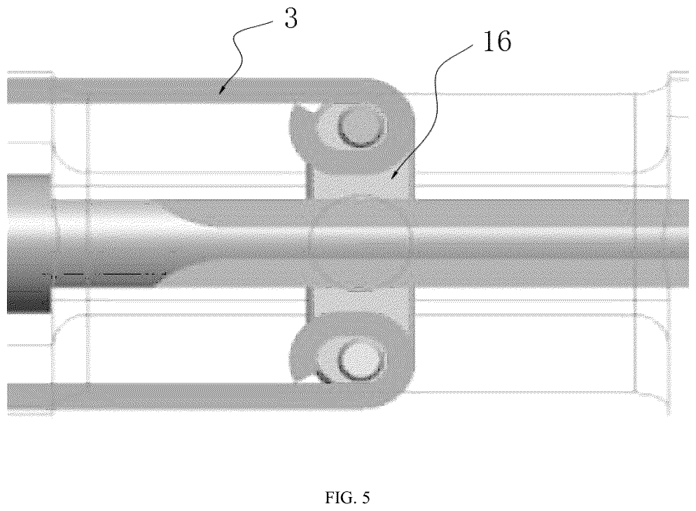

FIG. 5 is a schematic diagram of an internal structure before bending motion of the present disclosure;

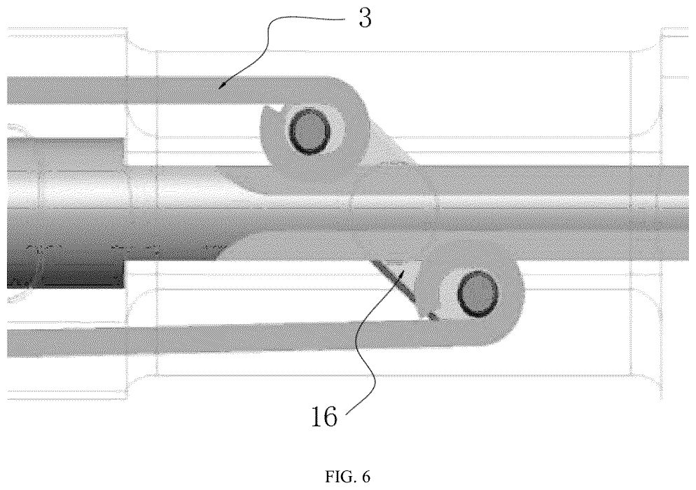

FIG. 6 is a schematic diagram of the internal structure after bending motion of the present disclosure.

In these FIGs: 1. anvil; 101. loading unit base; 2. cutter assembly; 3. steering pull rod; 4. inner sleeve cover; 5. inner sleeve; 6. trigger shaft; 7. sleeve connector; 8. propulsion rack; 9. outer sleeve; 10. steering sleeve; 11. connecting piece; 12. steering control seat; 13. steering connecting pin; 14. cutter steering seat; 15. connecting pin; 16. steering conversion seat; 17. steering linkage; 18. limit shaft.

### EMBODIMENT OF INVENTION

### Detailed Description

Technical solutions in embodiments of the present disclosure will be clearly and completely described with reference to the drawings in the embodiments of the present disclosure. Apparently, the embodiments described below are merely part, not all, of the embodiments of the present disclosure. Based on the embodiments described herein, all other embodiments obtained by those of ordinary skill in the art without creative work are within the scope of the present disclosure.

### Embodiment 1

As shown in FIG. 1-FIG. 4, the present disclosure provides the following technical solutions: An active bending system of a stapler comprises an anvil 1, steering pull rods 3, an inner sleeve 5, a sleeve connector 7, an outer sleeve 9, a connecting piece 11, a steering control seat 12, a steering conversion seat 16, a steering linkage 17 and a cutting mechanism, wherein the inner sleeve 5 is arranged in the outer sleeve 9 for limiting other parts;
the steering pull rods 3 are connected to a right side of the inner sleeve 5 for connecting the inner sleeve 5 and components in a stapler body; the steering conversion seat 16 is movably hinged in the steering pull rods 3 for pushing or pulling back the steering pull rods 3 to move left and right; the steering linkage 17 is arranged on the steering conversion seat 16 for connecting a steering power element of the stapler body and the steering conversion seat 16 to provide power for a bending function of the stapler; there are two steering pull rods 3 which are movably hinged on upper and lower sides of the steering conversion seat 16 respectively for pulling the steering control seat 12 to move left and right to realize a bending function of a stapler head; the steering control seat 12 is connected to the other end of the two steering pull rods 3, and the steering control seat 12 is rotationally connected to the inner sleeve 5 through a limit shaft 18 for circumferential motion around the limit shaft 18 to realize bending; a steering sleeve 10 is arranged on the outer sleeve 9 for limiting the anvil 1 and a loading unit base 101, to provide the stapler with initial clamping force to clamp tissue; and the anvil 1 and the loading unit base 101 are connected to a left side of the outer sleeve 9 through the steering sleeve 10, to provide the stapler with functions of tissue clamping and titanium pin forming.

Specifically, the cutting mechanism comprises a cutter assembly 2, an inner sleeve cover 4, a trigger shaft 6, and a propulsion rack 8; the cutter assembly 2, the inner sleeve cover 4, the trigger shaft 6 and the propulsion rack 8 are arranged in sequence from left to right; the inner sleeve cover 4 is clamped on the inner sleeve 5 for limiting the cutter assembly 2 and the trigger shaft 6 to ensure smooth operation; the cutter assembly 2 is arranged on a left side of the inner sleeve cover 4 for realizing a cutting function of the stapler and pushing the titanium pin to penetrate the tissue; the trigger shaft 6 is connected to a right side of the cutter assembly 2 for pushing or pulling back the cutter assembly 2; and the propulsion rack 8 is connected to a right side of the trigger shaft 6 for providing power for forward and backward movement of the cutter assembly 2 with a power gear connected to the stapler body and the trigger shaft 6.

Specifically, two steering connecting pins 13 are fixed on the steering control seat 12, and the two steering pull rods 3 are movably hinged with the steering control seat 12 through the two steering connecting pins 13 respectively.

Specifically, a cutter steering seat 14 is arranged in the inner sleeve 5, and the cutter steering seat 14 matches with the cutter assembly 2.

In this embodiment, the cutter steering seat 14 is set to limit the position and movement of the cutter assembly 2.

Specifically, the anvil 1 is rotationally connected with the steering control seat 12 through a connecting pin 15, and the loading unit base 101 is fixed with the steering control seat 12.

In this embodiment, a connection between the two steering pull rods 3 and the steering conversion seat 16 is of a waist-shaped hole structure.

Specifically, the bending system adopts a symmetrical design with two pull rods, and the connection between the two steering pull rods 3 and the steering conversion seat 16 is of the waist-shaped hole structure, as shown in FIG. 5 and FIG. 6. Only one steering pull rod 3 is used to pull the steering conversion seat 16 during bending or reset, and there are advantages of simple assembly and connection and symmetrical limit clearance. The steering conversion seat 16 moves flexibly and stably in the waist-shaped hole area, without resistance in cyclical switching of bidirectional driving and interference in linear movement with each other. One steering pull rod 3 generates thrust and one steering pull rod 3 generates tension, and the cooperation of thrust and tension makes the steering control seat complete bending movement and enables more accurate angle adjustment. The system is designed to pull the linkage to control the steering seat for active bending, effectively improving operation convenience, flexibility and stability.

The working principle and use flow of the present disclosure are as follows: The bending system is mainly divided into bending parts and power direction conversion parts, and see FIG. 4 for movement relationship: If the steering linkage 17 moves to the left, the steering control seat 12 will be driven to move counterclockwise, and if the steering linkage 17 moves to the right, the steering control seat 12 is driven to rotate about the limit shaft 18 in the inner sleeve 5, to realize rotation adjustment of the stapler head. After the stapler head is turned to the required position, the steering linkage 17 stops moving and is fixed to realize the positioning of the stapler head. The stapler can then start anastomosis. After the anastomosis is finished, the steering linkage 17 moves in reverse to pull the steering control seat 12 to rotate in reverse, completing the reset of the stapler head.

It should be finally noted that the embodiments above are only the preferred embodiments for the present disclosure, and not the restrictions to the present disclosure. Technicians in this art, despite the detailed descriptions of the present disclosure in the above embodiments, can still modify the technical solutions as mentioned in each of above embodiments, or replace some of technical features with equivalent ones. Any modification, equivalent replacement and improvement within the concept and principle of the present disclosure, are covered by the range of protection by the present disclosure.

1. An active bending system of a stapler, comprising an anvil (1), steering pull rods (3), an inner sleeve (5), a sleeve connector (7), an outer sleeve (9), a connecting piece (11), a steering control seat (12), a steering conversion seat (16), a steering linkage (17) and a cutting mechanism, wherein the inner sleeve (5) is arranged in the outer sleeve (9) for limiting other parts; the steering pull rods (3) are connected to a right side of the inner sleeve (5) for connecting the inner sleeve (5) and components in a stapler body; the steering conversion seat (16) is movably hinged in the steering pull rods (3) for pushing or pulling back the steering pull rods (3) to move left and right; the steering linkage (17) is arranged on the steering conversion seat (16) for connecting a steering power element of the stapler body and the steering conversion seat (16) to provide power for a bending function of the stapler; there are two steering pull rods (3) which are movably hinged on upper and lower sides of the steering conversion seat (16) respectively for pulling the steering control seat (12) to move left and right to realize a bending function of a stapler head; the steering control seat (12) is connected to the other end of the two steering pull rods (3), and the steering control seat (12) is rotationally connected to the inner sleeve (5) through a limit shaft (18) for circumferential motion around the limit shaft (18) to realize bending; a steering sleeve (10) is arranged on the outer sleeve (9) for limiting the anvil (1) and a loading unit base (101), to provide the stapler with initial clamping force to clamp tissue; and the anvil (1) and the loading unit base (101) are connected to a left side of the outer sleeve (9) through the steering sleeve (10), to provide the stapler with functions of tissue clamping and titanium pin forming.

2. The active bending system of the stapler according to claim 1, wherein the cutting mechanism comprises a cutter assembly (2), an inner sleeve cover (4), a trigger shaft (6), and a propulsion rack (8); the cutter assembly (2), the inner sleeve cover (4), the trigger shaft (6) and the propulsion rack (8) are arranged in sequence from left to right; the inner sleeve cover (4) is clamped on the inner sleeve (5) for limiting the cuter assembly (2) and the trigger shaft (6) to ensure smooth operation; the cutter assembly (2) is arranged on a left side of the inner sleeve cover (4) for realizing a cutting function of the stapler and pushing the titanium pin to penetrate the tissue; the trigger shaft (6) is connected to a right side of the cutter assembly (2) for pushing or pulling back the cutter assembly (2); and the propulsion rack (8) is connected to a right side of the trigger shaft (6) for providing power for forward and backward movement of the cutter assembly (2) with a power gear connected to the stapler body and the trigger shaft (6).

3. The active bending system of the stapler according to claim 2, wherein two steering connecting pins (13) are fixed on the steering control seat (12), and the two steering pull rods (3) are movably hinged with the steering control seat (12) through the two steering connecting pins (13) respectively.

4. The active bending system of the stapler according to claim 3, wherein a cutter steering seat (14) is arranged in the inner sleeve (5), and the cutter steering seat (14) matches with the cutter assembly (2).

5. The active bending system of the stapler according to claim 4, wherein the anvil (1) is rotationally connected with the steering control seat (12) through a connecting pin (15), and the loading unit base (101) is fixed with the steering control seat (12).

6. The active bending system of the stapler according to claim 5, wherein a connection between the two steering pull rods (3) and the steering conversion seat (16) is of a waist-shaped hole structure.
